(19) United States
(12) Patent Application Publication
(10) Pub. No.: US 2017/0000311 A1
(43) Pub. Date: Jan. 5, 2017

(54) STENT DELIVERY SYSTEM AND ENDOSCOPE SYSTEM

(71) Applicant: OLYMPUS CORPORATION, Tokyo (JP)

(72) Inventors: Toshihiro YAMAGATA, Tokyo (JP); Hiromichi MIYANO, Fujisawa-shi (JP)

(73) Assignee: OLYMPUS CORPORATION, Tokyo (JP)

(21) Appl. No.: 15/268,552

(22) Filed: Sep. 17, 2016

Related U.S. Application Data
(63) Continuation of application No. PCT/JP2015/053531, filed on Feb. 9, 2015.

(30) Foreign Application Priority Data
Mar. 26, 2014 (JP) 2014-063516

(51) Int. Cl. A61B 1/00; A61F 2/95; A61B 17/00; A61F 2/966

(52) U.S. Cl. CPC A61B 1/00; A61F 2/966; A61F 2/95; A61B 1/00133; A61B 17/00234; A61F 2230/0069

(57) ABSTRACT

A stent delivery system includes a guide catheter, a stent into which the guide catheter can be inserted, and a pusher catheter into which the guide catheter can be inserted and disposed in a proximal end side relative to the stent. The guide catheter has a guide catheter tip portion protruding to a distal end side further than the stent when the guide catheter is inserted into the stern and the pusher catheter, and a guide catheter body disposed at least partially in the passages of the stent and the pusher catheter. The stent has flexural rigidity equal to or lower than that of the guide catheter body, and the stent mounted region has flexural rigidity equal to or lower than that of the pusher mounted region.

### BACKGROUND OF THE INVENTION

Field of the Invention

The present invention relates to a stern delivery system and an endoscope system.

This application is a continuation application based on a PCT International Application No. PCT/JP2015/053531, filed on Feb. 9, 2015, whose priority is claimed on Japanese Patent Application No. 2014-063516, filed Mar. 26, 2014. Both of the content of the PCT International Application and the Japanese Application are incorporated herein by reference.

Description of Related Art

In the related art, a medical stent (hereinafter, also referred to simply as a “stent”) is indwelled into a stenosis formed in a lumen inside a living body, such as a blood vessel, a digestive tract, a bile duct, a pancreatic duct, or a urinary duct, in order to expand this stenosis and maintain an open state. In order to deliver (indwell) the stent into the stenosis, a guide catheter and a pusher catheter known in the art are employed. The stent, the guide catheter, and the pusher catheter constitute a stent delivery system (hereinafter, also referred to simply as “delivery system”). Such a type of delivery system is known in the art, for example, as disclosed in Japanese Unexamined Patent Application, First Publication No. 2006-204476.

The delivery system includes a guide catheter, a stent fitted onto an outer circumference of the guide catheter, and a pusher catheter fitted onto an outer circumference of the guide catheter and positioned closer to a hand side relative to the stent.

The delivery system is used as follows. A guide wire is introduced into the inside of a bile duct through a channel of an endoscope and is inserted until a tip thereof passes over the stenosis. Then, the delivery system provided with the stent and the pusher catheter disposed in a proximal end side of the stent is fitted to the guide wire from the hand side to the guide catheter, and the stent is forced to advance to the inside of the bile duct using the guide wire as a guide. Subsequently, sockets in the proximal end side of the guide catheter and the pusher catheter are disengaged, and the stent is pushed by the pusher catheter while keeping distal end positions of the guide catheter and the guide wire. In this case, the stent is inserted until a flap disposed in a rear end of the stent contacts with duodenal papilla. In this state, the stent is placed in the stenosis. This is because a medical operator selects a length of the stent appropriately depending on a patient in advance.

Subsequently, the pusher catheter is contacted with the stent to support the stent, and only the guide catheter is pulled back to the hand side while the stent is fixed immovably. As a result, the stent remains in a suitable position of the stenosis and is indwelled therein.

### SUMMARY OF THE INVENTION

According to a first aspect of the present invention, there is provided a stent delivery system including: a guide catheter that can be inserted into a channel of an endoscope; a stent formed in a tubular shape and provided with a first passage into which the guide catheter can be inserted; and a pusher catheter formed in a tubular shape, provided with a second passage into which the guide catheter can be inserted, and disposed in a proximal end side relative to the stent, the guide catheter having, in a state in which the guide catheter is inserted into the stent and the pusher catheter, a guide catheter tip portion protruding to a distal end side further than the stent, and a guide catheter body disposed inside the stent and the pusher catheter, wherein a portion of the guide catheter inserted into the stent is defined as a stein mounted region, a portion of the guide catheter inserted into the pusher catheter is defined as a pusher mounted region, the stent has flexural rigidity equal to or low that of the guide catheter body, and the stein mounted region has flexural rigidity equal to or lower than that of the pusher mounted region.

According to a second aspect of the present invention, the flexural rigidity of the guide catheter tip portion may be lower than that of the stent.

According to a third aspect of the present invention, the flexural rigidity of the stent may be lower than that of the guide catheter body.

According to a fourth aspect of the present invention, the pusher catheter may have a pusher tip and a pusher body positioned in a proximal end side of the pusher tip, and the flexural rigidity of the pusher tip may be lower than that of the pusher body.

According to a fifth aspect of the present invention, the stent may have an outer diameter equal to that of a distal end portion of the pusher catheter.

According to a sixth aspect of the present invention, while the guide catheter is inserted into the stent and the pusher catheter, the distal end side of the guide catheter body may protrude to the distal end side further than the stent.

According to a seventh aspect of the present invention, the guide catheter tip portion may have an outer diameter smaller than that of the guide catheter body.

According to an eighth aspect of the present invention, there is provided an endoscope system including: an endoscope having an insertion portion provided with a channel having an opening in a distal end portion and a bending section disposed in a proximal end side of the insertion portion relative to the opening and manipulated bendably; and the stent delivery system according to claim 4, that can be inserted into the channel, wherein, when at least part of the stent protrudes from the opening of the channel, at least part of the pusher tip is placed inside the bending section in an axial direction of the insertion portion.

### BRIEF DESCRIPTION OF THE DRAWINGS

FIG. 1 is a schematic diagram illustrating the entire configuration of an endoscope system according to a first embodiment of the invention.

FIG. 2 is a cross-sectional view illustrating main parts of the endoscope system.

FIG. 3 is aside view illustrating a delivery system of the endoscope system.

FIG. 4 is a cross-sectional view illustrating main parts of FIG. 3.

FIG. 5 is a diagram illustrating a three-point bending test method.

FIG. 6 is a cross-sectional view illustrating a side face of a proximal end side of the delivery system.

FIG. 7 is a diagram illustrating an operation of the endoscope system.

FIG. 8 is a diagram illustrating an operation of the endoscope system.

FIG. 9 is a diagram illustrating a state in which a stent indwells into a delivery system of the related art.

FIG. 10 is a cross-sectional view illustrating main parts of a delivery system according to a second embodiment of the invention.

FIG. 11 is a cross-sectional view illustrating operations of the delivery system and the endoscope.

FIG. 12 is a cross-sectional view illustrating main parts of the delivery system according to a modification of the second embodiment of the invention.

FIG. 13 is a cross-sectional view illustrating main parts of a pusher catheter of the delivery system according to a modification of the second embodiment of the invention.

FIG. 14 is a cross-sectional view illustrating main parts of the delivery system according to a third embodiment of the invention.

### DETAILED DESCRIPTION OF THE INVENTION

### First Embodiment

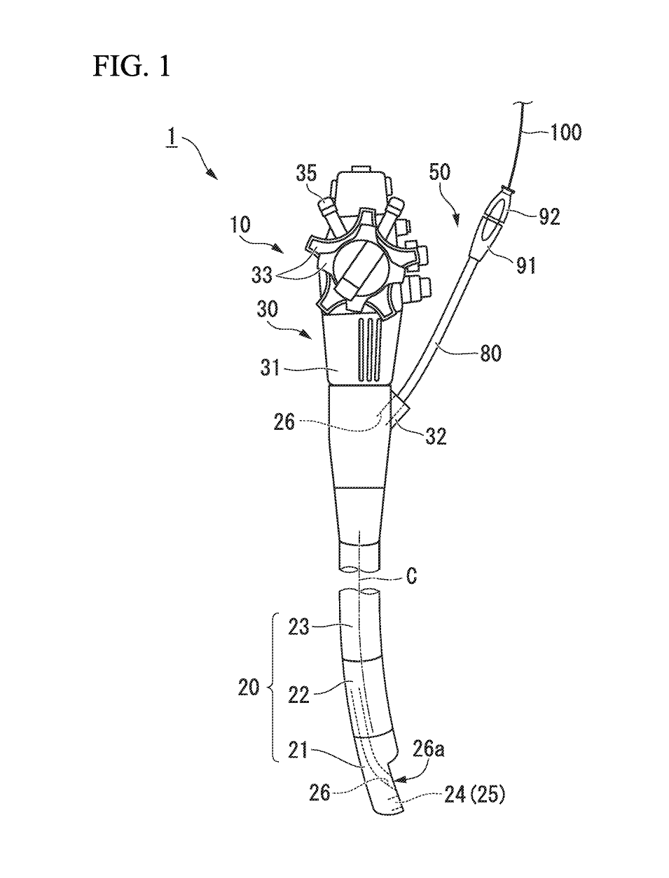

An endoscope system according to a first embodiment of the invention will now be described with reference to FIGS. 1 to 9. As illustrated in FIG. 1, the endoscope system 1 according to this embodiment includes an endoscope 10 provided with a long insertion portion 20 and a delivery system 50 that can be inserted into a channel 26 formed in the insertion portion 20. Hereinafter, an insertion portion 20 side with respect to a manipulation unit 30 to be described later will be referred to as a “distal end side,” and a manipulation unit 30 side with respect to the insertion portion 20 will be referred to as a “proximal end side.”

The endoscope 10 is a so-called flexible side-view type endoscope. The endoscope 10 has the insertion portion 20 described above and the manipulation unit 30 provided in a proximal end portion of the insertion portion 20. The insertion portion 20 has a hard tip section 21 provided in a distal end portion, a bending section 22 provided in a proximal end side of the hard tip seam and manipulated bendably, and a flexible tube section 23 provided in a proximal end side of the bending section 22. On the side face of the hard tip section 21, a tip portion of a light guide 24 and an image sensing unit 25 provided with charge coupled devices (CCDs) (not shown) are provided in an exposed state. In the insertion portion 20, the channel 26 described above is formed in an axial line C direction of the insertion portion 20. A distal end portion of the channel 26 is opened to the aforementioned side face of the hard tip section 21.

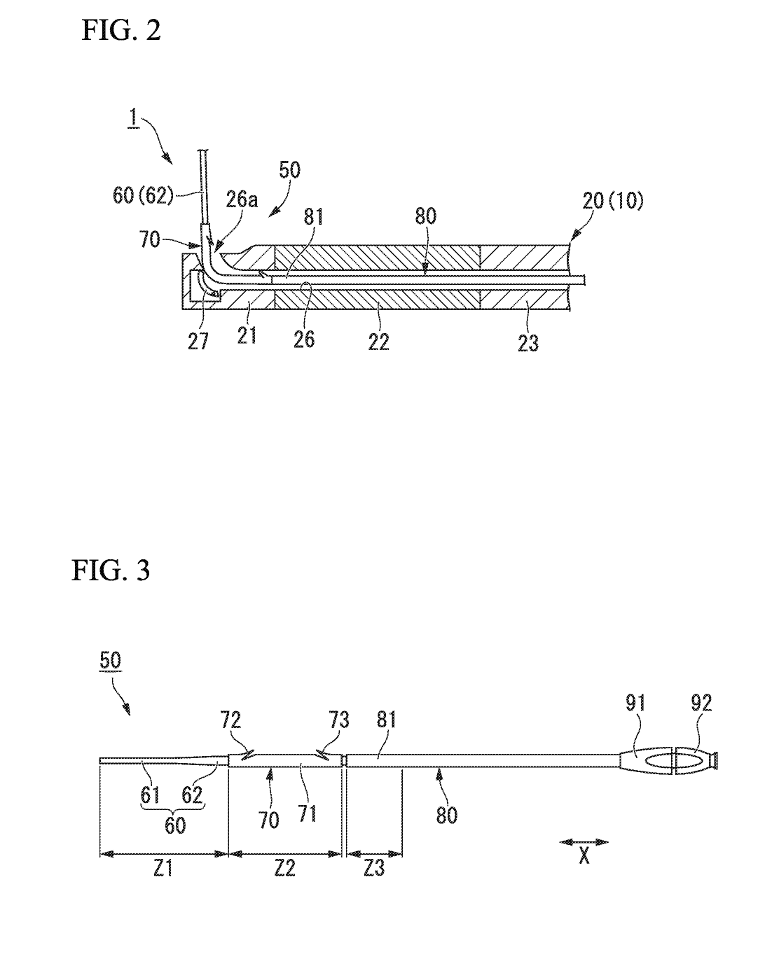

A portion of the channel 26 corresponding to the hard tip section 21 is provided with a raising base 27 as illustrated in FIG. 2. A shape of the channel 26 around the raising base 27 is bent. A raising base control wire (not shown) fixed to th raising base 27 extends to the proximal end side through the inside of the insertion portion 20.

The bending section 22 is internally provided with a plurality of bending pieces (not illustrated) arranged side by side in the axial line C direction of the insertion portion 20 and swingably connected to each other. A tip of the angulation control wire (not shown) is fixed to the outermost one of the angulation bands arranged on the distal end side. The bending section 22 is disposed closer to the proximal end side relative to the opening 26a of the distal end side of the channel 26. The angulation control wire extends to the proximal end side through the inside of the insertion portion 20.

As illustrated in FIG. 1, a forceps outlet 32 is provided in the distal end side of the control body 31 of the manipulation unit 30. The proximal end portion of the channel 26 is opened to the forceps outlet 32. A knob 33 for controlling the angulation control wire described above and a lever 35 for controlling the raising base control are provided in the proximal end side of the control body 31. By manipulating the knob 33, the bending section be bent in a desired direction. By manipulating the lever 35, an angle of the raising base 27 can be changed.

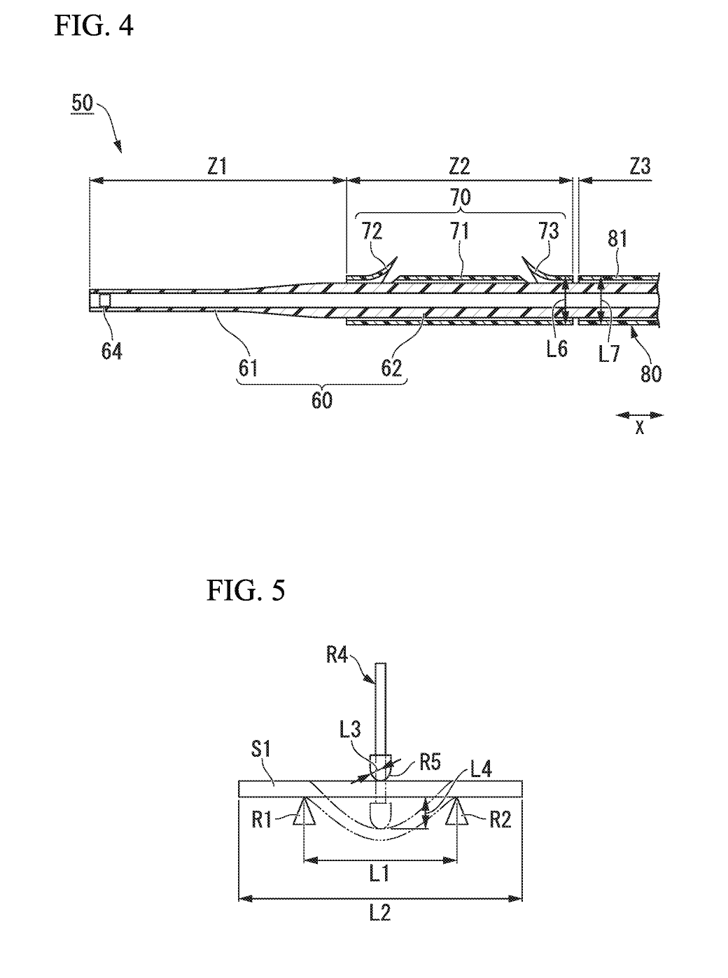

Next, the delivery system 50 will be described. As illustrated in FIGS. 3 and 4, the delivery system 50 includes a guide catheter 60 that can be inserted into the channel 26 of the endoscope 10, a stent 70 formed in a tubular shape and provided with a passage (first passage) where the guide catheter 60 can be inserted, and a pusher catheter 80 formed in a tubular shape, provided with a passage (second passage) into which the guide catheter 60 can be inserted, and arranged in a proximal end side relative to the stent 70.

With respect to the guide catheter 60, the stent 70 and the pusher catheter 80 can move in the longitudinal direction X of the guide catheter 60 while they slide along the outer circumferential surface of the guide catheter 60. A target placement state of the stent 70 and the pusher catheter 80 with respect to the guide catheter 60 is defined as a state where the entire guide catheter tip portion 61 described below and the distal end side of the guide catheter body 62 protrude as illustrated in FIGS. 3 and 4. In addition, in the delivery system 50, the thicknesses of the stent 70 and the pusher catheter 80 are relatively reduced. A clearance between the guide catheter 60 and the stent 70 or the pusher catheter 80 is relatively reduced. An outer diameter of the guide catheter 60 increases. Note that, herein, the “thickness” refers to a radial dimension of the tube wall in a tubular structure.

Here, in the longitudinal direction X, a portion of the guide catheter 60 protruding from the stent 70 to the distal end side is referred to as a “guide catheter distal end region Z1,” a portion of the guide catheter 60 inserted into the passage of the stent 70 is referred to as a “stent mounted region Z2,” and a portion of the guide catheter 60 inserted into the passage of the distal end portion 81 of the pusher catheter 80 is referred to as a “pusher mounted region Z3.” Here, the distal end portion 81 of the pusher catheter 80 ranges from the opening 26a of the channel 26 of the endoscope 10 to the bending section 22 of the endoscope 10 in the process of indwelling the stent 70 described below.

Here, a three-point bending test for measuring flexural rigidity will be described along with the description of each configuration of the delivery system 50. As illustrated in FIG. 5, a pair of fulcrums R1 and R2 is arranged apart from each other along the horizontal plane. The distance L1 between the fulcrums R1 and R2 is set to 30 mm. A sample S1 including a guide catheter tip portion 61, a guide catheter body 62, the stent 70, and the pusher catheter 80 described below is prepared to have a length L2 of, for example, approximately 80 mm. For example, if the length of the stent 70 is shorter than 80 mm, a sample S1 having the same outer diameter, inner diameter, and material as those of the stent 70 but having a length of 80 mm is used. The length L2 of the sample S1 is set such that the sample S1 is not separated from the fulcrums R1 and R2 even by pushing and bending a longitudinal center of the sample S1. The sample S1 is disposed on the fulcrums R1 and R2 such that a protruding length of one end of the sample S1 from the fulcrum R1 is the same as a protruding length of the other end of the sample S1 from the fulcrum R2.

A contact surface R5 with which a pusher R4 that pushes the sample S1 downward comes into contact is curved with a radius of curvature L3 of 5 mm to distribute a load exerted on the sample S1 so that it is not concentrated on a particular point. The sample S1, the fulcrums R1 and R2 as test jig, and the pusher R4 comply with a plastic flexural test method of the standard JIS K7171. The pusher R4 is set such that the contact surface R5 of the pusher R4 comes in contact with a top surface of the longitudinal center of the sample S1. As the pusher R2 comes in contact with the sample S1, and a downward pushing force is exerted up to a pushing distance L4 at a pushing speed of 5 mm/min, a maximum reactive force is measured in the meantime. The maximum reactive force is defined as flexural rigidity of the sample S1. Note that the pushing distance L4 is set to 5 mm. Here, the flexural rigidity is measured based on this three-point bending test.

As illustrated in FIGS. 3 and 4, in a target placement state in which the guide catheter 60 is inserted into passages of the stent 70 and the pusher catheter 80, the guide catheter 60 has a guide catheter tip portion 61 protruding to the distal end side from the stern 70, and a guide catheter body 62 disposed in th passages of th stent 70 and the pusher catheter 80. That is, a distal end portion of the guide catheter 60 is the guide catheter tip portion 61, and a portion of the guide catheter 60 extending from the guide catheter tip portion 61 to the proximal end side is the guide catheter body 62.

The guide catheter tip portion 61 has an outer diameter smaller than that of the guide catheter body 62. The guide catheter tip portion 61 is thinned and trimmed by grinding a pipe material of the guide catheter body 62 or through heated drawing. As a result, it is possible to obtain optimum flexural rigidity as the guide catheter tip portion 61. The guide catheter tip portion 61 is tapered such that its outer diameter is reduced smoothly toward the distal end side from a connecting portion with the guide catheter body 62, so that the flexural rigidity is smoothly reduced accordingly. If the guide catheter 60 has a large outer diameter and high flexural rigidity, the tip portion of the guide catheter 60 becomes hard. As a result, a following property of the guide catheter 60 to the guide wire may be degraded, which causes difficulty, or an undesirable burden may be given to a human body during an approach to duodenal papilla. In this regard, according to this embodiment, the guide catheter tip portion 61 is formed in a tapered shape to prevent such difficulty or burden.

A tubular X-ray radiopacity marker 64 is provided in a distal end side of the guide catheter tip portion 61. The outer and inner diameters and the flexural rigidity of the guide catheter body 62 are constant regardless of a position in the longitudinal direction X. The guide catheter tip portion 61 and the guide catheter body 62 are formed of, for example, a resin material having high flexural rigidity and excellent biological compatibility, such as a susethylenetetrafluoride-propylenehexafluoride copolymer (FEP), a copolymer resin of tetrafluoroethylene and a perfluoroalkyl vinylether (PEA), and polyvinylidene fluoride (PVDF), and are integrated with each other in a tubular shape. The resin material such as FEP, PFA, and PVDF are advantageous in high flexural rigidity and low sliding friction.

The stent 70 has a stent body 71 formed in a tubular shape and flaps 72 and 73 provided in a distal end portion and a proximal end portion, respectively, of the stent body 71. The flap 72 is opened to the outside of the radial direction toward the proximal end side. The flap 73 is opened to the outside of the radial direction toward the distal end side. In this example, the flaps 72 and 73 are formed by cutting and elevating a proximal end side and a distal end side of a tubular member, so that the flaps 72 and 73 and the stent body 71 are formed as an integral manner. The flexural rigidity of the stent 70 is equal to or lower than that of the guide catheter body 62.

The stent 70 preferably has a thin thickness. As the clearance between the guide catheter 60 and the stent 70 or the pusher catheter 80 is reduced, and the outer circumference of the guide catheter 60 can increase, it is possible to optimize the flexural rigidity of the guide catheter 60.

Generally, a stent is required to have tradeoff relationship between bendability, that is, flexibility to follow a curved shape of a bile duct in a living body or a motion of the living body, and stiffness (lumen holdability) for holding a size of its lumen (passage) without being deformed when the stent is bent. If the lumen is deformed and narrowed by bending of the stent in a living body, it may be difficult to deliver bile or the like, which may cause a significant problem. Typically, a stent having a wide lumen is not as easily occluded as a stent having a narrow lumen. Therefore, it is preferable that the stent have a wide lumen. In this regard, as discussed in Japanese Patent Publication No. 4981994, a coil may be provided between the inner and outer layers. As a result, it is possible to obtain a stent having a wide lumen with a thin thickness.

According to this embodiment, the pusher catheter 80 is formed from a single-layer tube having constant outer and inner diameters and the same material property regardless of a position in the longitudinal direction X. That is, the flexural rigidity of the pusher catheter 80 is constant regardless of a position in the longitudinal direction X. Since, in the longitudinal direction X, a contact length between the pusher catheter 80 and the guide catheter 60 is longer than a contact length between the stent 70 and the guide catheter 60, it is necessary to reduce sliding friction exerted between the pusher catheter 80 and the guide catheter 60. If the guide catheter 60 is formed of a fluororesin, it is preferable that the pusher catheter 80 be formed of a material other than the fluororesin, for example, an elastomer such as polyethylene (PE), polypropylene (PP), an olefin, or an amide. Similar to the guide catheter 60, the stent 70 and the pusher catheter 80 are also formed to have an outer diameter that can be inserted into the channel 26 of the endoscope 10.

The stent mounted region Z2 has flexural rigidity equal to or lower than that of the pusher mounted region Z3. In order to adjust the flexural rigidity in this manner, for example, the flexural rigidity of each of the stent 70 and the pusher catheter 80 may be adjusted individually. That is, the flexural rigidity may be adjusted by changing materials or structures of the stent 70 and the pusher catheter 80 (by providing a double layer structure or providing a double layer structure with a reinforcement layer interposed therebetween). Not limited thereto, the flexural rigidity may be adjusted, for example, by changing inner diameters or thicknesses of the stent 70 and the pusher catheter 80. The delivery system 50 having the aforementioned structure in a target placement state is configured to maintain the same flexural rigidity or increase the flexural rigidity without being decreased from the guide catheter distal end region Z1 to the proximal end side, through the stent mounted region Z2 and the pusher mounted region Z3.

The thickness of the pusher catheter 80 is reduced as much as the clearance is reduced as described above. Since the pusher catheter 80 is pushed from the forceps outlet 32 side of the endoscope 10, it is preferable that the flexural rigidity be high in order to effectively transmit the pushing force to the distal end side. On the other hand it is preferable that the flexural rigidity of the pusher catheter 80 be low in order to pass through the curved channel 26 of the endoscope 10. As illustrated in FIG. 4, the outer diameter L6 of the stent 70 is (approximately) the same as the outer diameter L7 of the distal end portion 81 of the pusher catheter 80. The stent 70 and the pusher catheter 80 can move in the longitudinal direction X relative to the guide catheter 60. As the proximal end portion of the stent 70 abuts on the distal end portion 81 of the pusher catheter 80, the pusher catheter 80 restricts movement of the stent 70 toward the proximal end side.

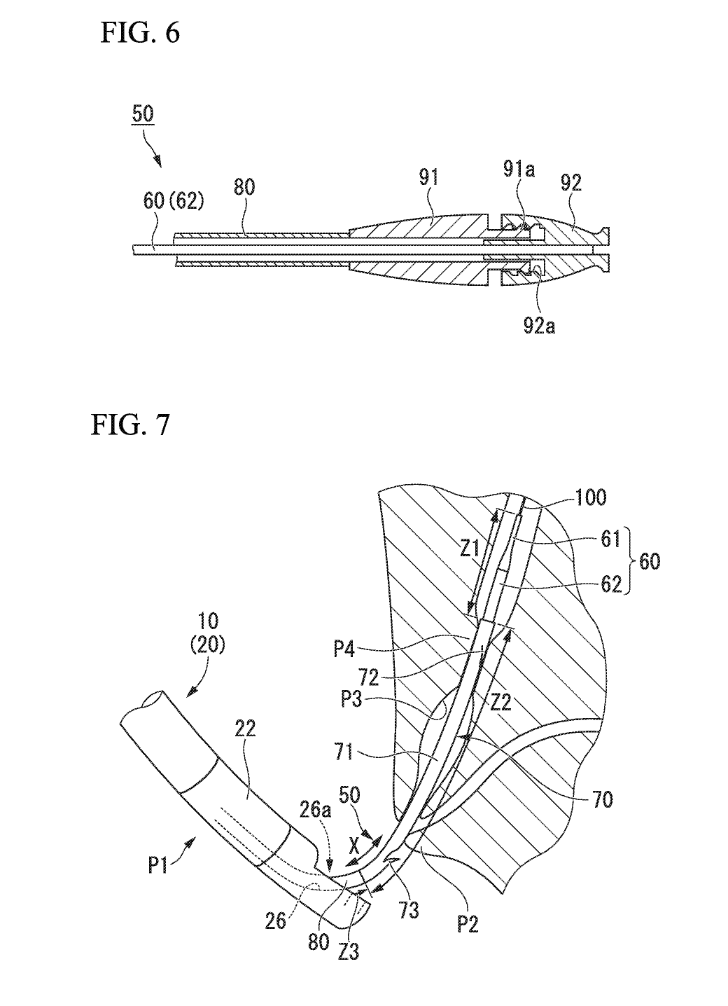

As illustrated in FIG. 6, the proximal end portion of the pusher catheter 80 is provided with a pusher socket 91. A male thread 91a is formed on the proximal end portion of the pusher socket 91. A socket 92 is provided in the proximal end portion of the guide catheter body 62 of the guide catheter 60. The distal end portion of the socket 92 is provided with a be ale thread 92a screwed onto the male thread 91a.

Next, operations of the endoscope system 1 configured as described above will be described, for example, assuming that the stent 70 indwells into a bile duct. As the light source of the endoscope 10 is operated, illumination light emitted from the light source is guided by the light guide 24 to illuminate the vicinity of the hard tip section 21 of the insertion portion 20. The image of the vicinity of the hard tip section 21 obtained from the image sensing unit 25 is displayed on a monitor. A user inserts the insertion portion 20 of the endoscope 10 into a coelom of a patient from a natural orifice such as a mouth while monitoring the image displayed on the monitor. In this case, a user bends the bending section 22 as necessary by manipulating the knob 33.

As illustrated in FIG. 7, a distal end portion of the insertion port on 20 advances to the vicinity of the duodenal papilla P2 through the duodenum P1. The opening 26a of a distal end side of the channel 26 faces the duodenal papilla P2. The guide wire 100 is inserted from the forceps outlet 32 of the endoscope 10. The guide wire protruding from the opening 26a of the channel 26 is inserted into the stenosis P4 of the bile duct P3.

Then, the raising base 27 of the endoscope 10 is set to a full-up state. The delivery system 50 includes the guide catheter 60 which the stent 70 and the pusher catheter 80 disposed in the proximal end side of the stent 70 are fitted to, the delivery system 50 covers the guide wire 100from the hand side of the guide wire 100, and the tip portion of the guide catheter 60 is inserted into the forceps outlet 32 of the endoscope 10. The tip of the guide catheter 60 contacts with the raising base 27 of the endoscope 10, the raising base 27 is set to a down state, and the delivery system 50 advances inside the bile duct P3 using the guide wire 100 as a guide. In this case, the delivery system 50 is inserted into the stenosis P4 of the bile duct P3 by repeating a cooperative manipulation between a manipulation of the raising base 27 using a suitable lever 35 or a manipulation of the bending section 22 using the knob 33 and an alternating manipulation of pushing and pulling the delivery system 50 from the forceps outlet 32 side of the endoscope 10. Then, the engagement between the socket 92 of the proximal end side of the guide catheter 60 and the pusher socket 91 of the distal end side of the pusher catheter 80 is released. While distal end positions of the guide catheter 60 and the guide wire 100 are maintained, the stent 70 is pushed using the pusher catheter 80. In this case, the stent 70 is pushed to be advanced until the flap 73 provided in the proximal end portion of the stent 70 contacts with the duodenal papilla P2.

In this case, this process may be performed while a position of the tip portion of the guide catheter 60 is recognized by monitoring a position of the X-ray radiopacity marker 64 under an X-ray radiation environment. As illustrated in FIG. 7, the stent 70 is inserted into a desired position while the distal end positions of the guide catheter 60 and the guide wire 100 are constantly maintained. In this case, the distal end portion of the guide catheter body 62 is preferably inserted into the deeper side over the stenosis P4. In addition, since the outer diameter L6 of the stent 70 is equal to the outer diameter L7 of the distal end portion 81 of the pusher catheter 80, a force exerted the pusher catheter 80 is reliably transmitted to the stent 70.

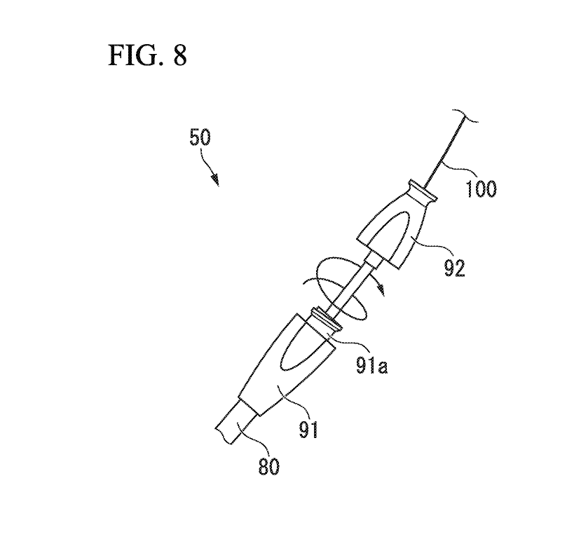

Then, as illustrated in FIG. 8, the socket 92 is removed from the pusher socket 91 by rotating the socket 92 with respect to the pusher socket 91. The distal end of the guide catheter 60 is extracted to the vicinity of the raising base 27 of the endoscope 10 by pulling the socket 92 while holding the pusher socket 91. As a result, the stent 70 indwells into the bile duct P3. Finally, the remaining parts of the delivery system 50 other than the stent 70 are extracted from the forceps outlet 32 of the endoscope 10 by gripping the proximal end side of the pusher catheter 80.

Through the aforementioned process, the flaps 72 and 73 of the stent 70 are inserted into locking positions of the stenosis P4 and the duodenal papilla P2, respectively. In this case, the entire guide catheter tip portion 61 and the distal end portion of the guide catheter body 62 protrude to the distal end side further than the stent 70, and the stent 70 and the pusher catheter 80 become the target placement state with respect to the guide catheter 60, so that the guide catheter distal end region Z1, the stent mounted region Z2, and the pusher mounted region Z3 are formed. Therefore, the flexural rigidity is maintained constantly or is increased without being decreased from the guide catheter distal end region Z1 to the pusher mounted region Z3 through the stent mounted region Z2. For this reason, it is possible to suppress formation of a so-called flexural rigidity dropping portion caused by an increase of the flexural rigidity in both the distal and proximal end sides relative to its flexural rigidity.

Therefore, when the pusher catheter 80 is shifted (pushed) to the distal end side with respect to the guide catheter 60, the delivery system 50 is not bent with a small radius of curvature, but is curved in a “U-shape” with a relatively large radius of curvature. That is, it is possible to distribute stress applied to the aligned portion in the longitudinal direction between the stent 70 and the pusher catheter 80. For this reason, a pushing force of the pusher catheter 80 is effectively transmitted to the stent 70. Then, the guide catheter 60, the pusher catheter 80, and the guide wire 100 are pulled out together from the bile duct P3 and are extracted from the channel 26 of the endoscope 10, so that the stent 70 indwells (is released) into the bile duct P3.

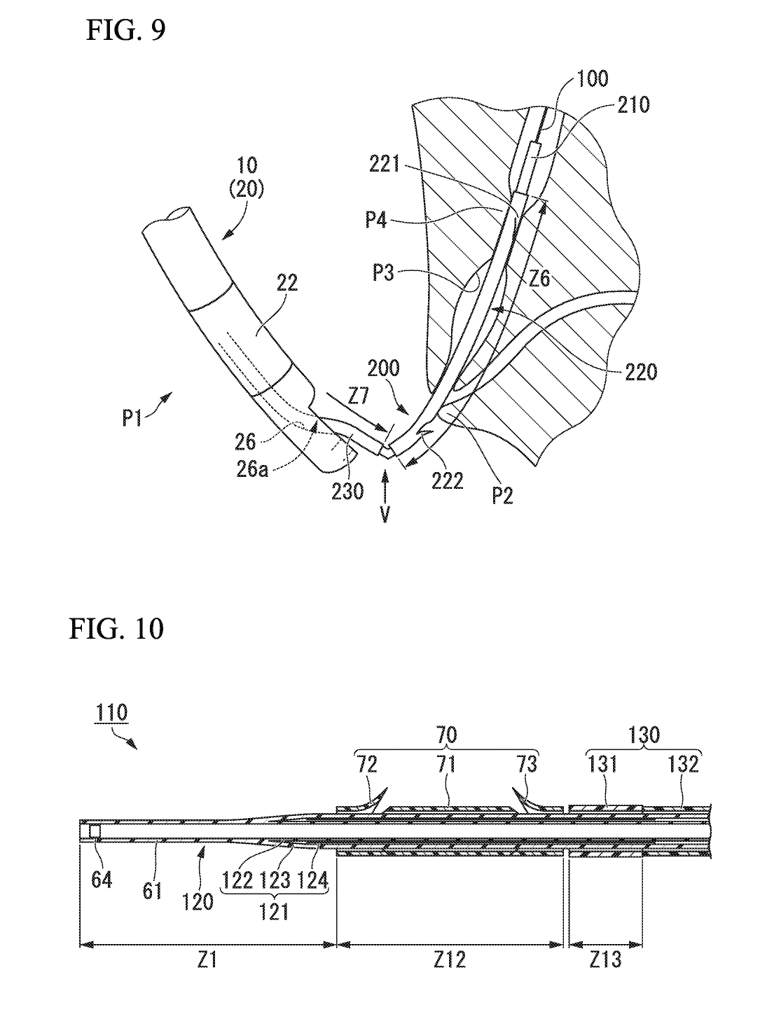

FIG. 9 illustrates a process of inserting a delivery system 200 of the related art into a channel 26 of the endoscope 10 and indwelling a stent into a bile duct P3. The delivery system 200 includes a guide catheter 210, a stent 220, and a pusher catheter 230. The guide catheter 210, the stent 220, and the pusher catheter 230 are formed in a tubular shape. The stent 220 is provided with flaps 221 and 222.

The guide catheter 210 of the related art has constant flexural rigidity regardless of a longitudinal position. The flexural rigidity of the guide catheter 210 is higher than that of the stent 220. The flexural rigidity of the stent 220 is lower than that of the pusher catheter 230. Since the guide catheter 210 is disposed inside the passages of the stent 220 and the pusher catheter 230, the guide catheter 210 has an outer diameter smaller than that of the stent 220 or the pusher catheter 230. The flexural rigidity of guide catheter 210 tends to decrease relative to the flexural rigidity of the stent 220 or the pusher catheter 230. In addition, since a distal end portion of the guide catheter 210 is inserted into the inside of a bile duct along with the guide wire, it is preferable that the flexural rigidity of the guide catheter 210 be small n order to reduce a burden on a patient.

If the guide catheter 210, the stent 220, and the pusher catheter 230 have the flexural rigidity as described above, a region Z6 in which the guide catheter 210 having low flexural rigidity is covered by the stent 220 and a region Z7 in which the guide catheter 210 having low flexural rigidity is covered by the pusher catheter 230 having high flexural rigidity are formed in the aligned portion between the stent 220 and the pusher catheter 230. That is, a flexural rigidity dropping portion (where the flexural rigidity is relatively smaller) is formed between the regions Z6 and Z7 in the longitudinal direction. Therefore, when the pusher catheter 230 is pushed toward the guide catheter 210, a sole portion of the guide catheter 210 in a free space V between the opening 26a of the channel 26 and the duodenal papilla P2 is bent in a “V-shape” with a small radius of curvature. For this reason, the pushing force of the pusher catheter 230 is not effectively transmitted to the stent 220.

In the delivery system 50 according to the first embodiment, the flexural rigidity of the stent 70 is equal to or lower than that of the guide catheter body 62, and the flexural rigidity of the stent mounted region Z2 is equal to or lower than that of the pusher mounted region Z3. For this reason, in the delivery system 50 having the target placement state, the flexural rigidity is constantly maintained or increases without being decreased toward the proximal end side. Therefore, it is possible to suppress formation of a flexural rigidity dropping portion in the aligned portion in the longitudinal direction X between the stent 70 and the pusher catheter 80 and suppress the aligned portion in the longitudinal direction from being bent with a small radius of curvature. Compared to the technique of the related art, it is possible to easily insert and indwell the stent 70 into a desired position with a smaller force and reduce a burden on an operator and a patient. Furthermore, since a force for pushing the pusher catheter 80 is reduced compared to the technique of the related art, a stent having a larger outer diameter can also be inserted.

Since the outer diameter L6 of the stent 70 is equal to the outer diameter L7 of the distal end portion 81 of the pusher catheter 80, it is possible to reliably transmit a force exerted on the pusher catheter 80 to the stent 70 and reliably push the stent 70 by the pusher catheter 80. In the target placement state, the distal end side of the guide catheter body 62 protrudes to the distal end side further than the stent 70. For this reason, even when a position of the stent 70 with respect to the guide catheter 60 slightly deviates toward the distal end side, it is possible to constantly maintain or increase the flexural rigidity of the delivery system 50 toward the proximal end side. Since the outer diameter of the guide catheter tip portion 61 is smaller than the outer diameter of the guide catheter body 62, it is possible to easily insert the guide catheter tip portion 61 of the guide catheter 60 into the bile duct P3.

According to the first embodiment, the guide catheter tip portion 61 may have flexural rigidity lower than that of the stent 70.

According to the first embodiment, the flexural rigidity of the stent 70 may not be equal to that of the guide catheter body 62, but may be lower than that of the guide catheter body 62. In this configuration, the flexural rigidity increases toward the proximal end side in the stent mounted region Z2 and the pusher mounted region Z3. Therefore, it is possible to more reliably suppress the aligned portion between the stent 70 and the pusher catheter 80 from being bent with a small radius of curvature. In the target placement state of the pusher catheter 80 with respect to the guide catheter 60, part of the distal end side of the guide catheter body 62 protrudes to the distal end side further than the stent 70 as described above. However, in the target placement state described above, the guide catheter body 62 may be configured not to protrude toward the distal end side further than the stent 70.

### Second Embodiment

Next, a second embodiment of the present invention will be described with reference to FIGS. 10 to 13, in which like reference numerals denote like elements, and only differences thereof will be described for the purpose of simplicity. In the first embodiment, if the stent 70 has an outer diameter of approximately 11.5 Fr (where 1 Fr is ⅓ mm), the outer diameter of the guide catheter 60 increases, and the flexural rigidity of the guide catheter 60 excessively increases. In this regard, the guide catheter may be configured to have a multi-layer tube structure having inner and outer layers formed in a tubular shape and stacked in a radial direction in order to reduce the flexural rigidity of the guide catheter. The inner and outer layers are bonded, for example, only on both ends. In this case, a reinforcement layer may be provided in a gap between the inner and outer layers to adjust the flexural rigidity.

Generally, similar to the stent, the guide catheter is required to have a tradeoff relationship between bendability, that is, flexibility to follow a curved shape of a bile duct in a living body, and stiffness (lumen holdability) for holding a size of its lumen (passage) without being deformed or folded in the outer layer when the guide catheter is bent. If the lumen of the guide catheter is deformed, and is folded in the outer layer when the guide catheter is bent in the course of insertion of the stent, a force for pushing and advancing the stent or pulling the guide catheter may increase, or a lumen of the stem may be damaged disadvantageously.

In order to address such problems, the delivery system 110 according to the second embodiment is provided with a guide catheter 120 and a pusher catheter 130 as illustrated in FIG. 10 instead of the guide catheter 60 and the pusher catheter 80 of the delivery system 50 of the first embodiment.

The guide catheter body 121 of the guide catheter 120 has an inner layer 122 and an outer layer 123 formed in a tubular shape and a reinforcement layer 124 interposed between the inner layer 122 and the outer layer 123. The inner layer 122 and the outer layer 123 are disposed inside the passage. The guide catheter tip portion 61 and the inner layer 122 and the outer layer 123 are formed of the same material as the guide catheter 60 in an integral manner The reinforcement layer 124 is formed in a coil shape. Note that the reinforcement layer 124 may be formed as a blade having a mesh shape using metal or resin strands instead of the coil. The inner layer 122 and the outer layer 123 and the reinforcement layer 124 are arranged in a coaxial state. A proximal end portion of the reinforcement layer 124 extends to the proximal end side further than a proximal end portion of a pusher tip 131 to be described below.

The pusher catheter 130 has a pusher tip 131 and a pusher body 132 positioned in the proximal end side of the pusher tip 131. That is, a distal end portion of the pusher catheter 130 is the pusher tip 131, and a portion of the pusher catheter 130 provided in the proximal end side relative to the pusher tip 131 is the pusher body 132. The pusher tip 131 has flexural rigidity lower than that of the pusher body 132. In order to adjust the flexural rigidity in the pusher tip 131 and the pusher body 132, for example, the pusher tip 131 may be formed of a relatively soft polyamide elastomer, and the pusher body 132 may be formed of a relatively hard polyamide. Alternatively, the pusher tip 131 and the pusher body 132 may be formed of the same material, and the pusher tip 131 may have a thickness thinner than that of the pusher body 132. Alternatively, the pusher tip 131 and the pusher body 132 may be formed by bonding a pair of tubes having different flexural rigidity by heat welding.

For example, in the pusher catheter 130 of the delivery system having an outer diameter of approximately 10 Fr or larger, it is preferable that the flexural rigidity of the pusher tip 131 be different from the flexural rigidity of the pusher body 132. Note that the flexural rigidity decreases as the outer diameter decreases. For this reason, if the outer diameter of the pusher catheter is equal to or smaller than a predetermined value, stress is not concentrated on the aligned portion in the longitudinal direction between the stent and the pusher catheter even when a pair of tubes having different flexural rigidity is not used.

Similarly, according to the second embodiment, a stent mounted region Z12 formed by inserting the guide catheter 120 into the passage of the stent 70 and a pusher mounted region Z13 formed by inserting the guide catheter 120 into the passage of the pusher tip 131 of the pusher catheter 130 are set. By adjusting the flexural rigidity in the guide catheter 120, the stent 70, and the pusher catheter 130, the stent 70 has flexural rigidity equal to or lower than that of the guide catheter body and the stent mounted region Z12 has flexural rigidity equal to or lower than that of the pusher mounted region Z13.

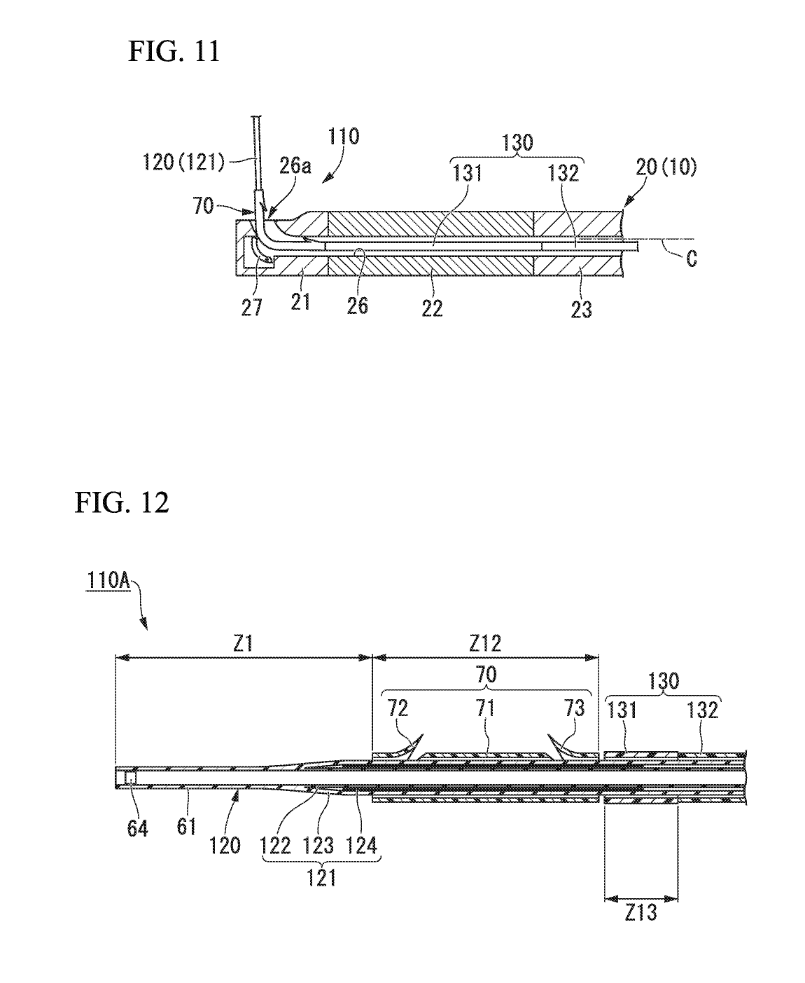

Operations of the delivery system 110 configured as described above will be described. As illustrated in FIG. 11, when the pusher catheter 130 is pushed to the guide catheter 120, a distal end portion of the stent 70 protrudes from the opening 26a of the channel 26. In this case, at least part of the pusher tip 131 is located inside the bending section 22 in the axial line C direction of the insertion portion 20. Since the flexural rigidity of the pusher tip 131 is lower than that of the pusher body 132, it is possible to relatively easily perform a bending manipulation of the bending section 22 even when the pusher catheter 130 is disposed inside the channel 26 formed in the bending section 22.

As described above, using the delivery system 110 and the endoscope system according to the second embodiment, it is possible to suppress the aligned portion between the stent 70 and the pusher catheter 130 from being bent with a small radius of curvature.

According to the second embodiment, when the entire stent 70 protrudes to the outside from the opening 26a of the channel 26, part of the pusher tip 131 may overlap part of the bending section 22 in the axial line C direction. Even in this configuration, the same advantages as those of the second embodiment can be obtained. In addition, as illustrated in FIG. 12, the delivery system 110A may be configured such that the proximal end portion of the reinforcement layer 124 extends to an intermediate portion of the pusher tip 131 in the longitudinal direction.

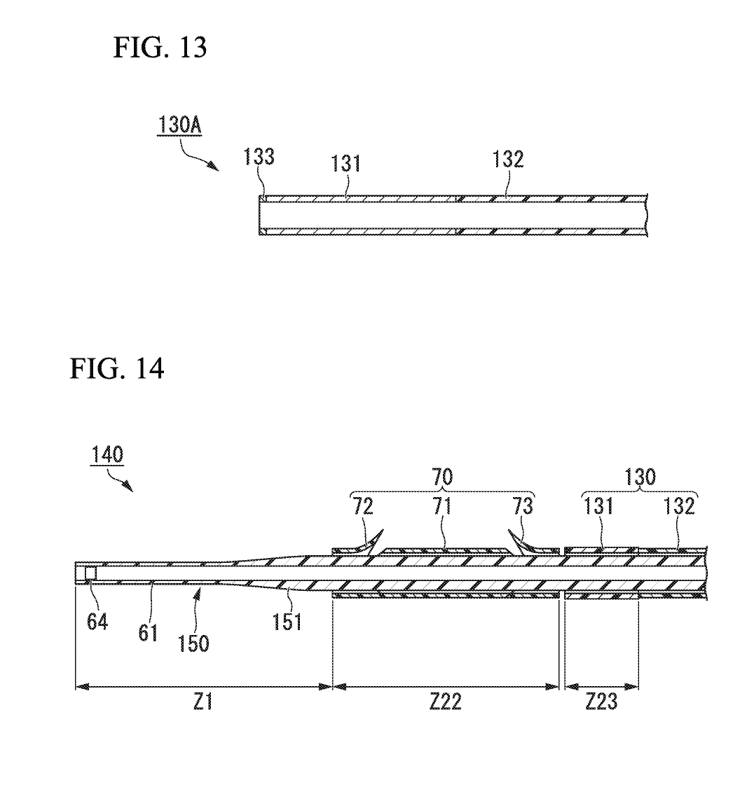

As illustrated in FIG. 13, a hard portion 133 may be provided in the distal end side of the pusher tip 131 of the pusher catheter 130A. The hard portion 133 may have flexural rigidity higher than that of the pusher tip 131. The flexural rigidity of the hard portion 133 is preferably equal to that of the push body 132. By providing the hard portion 133 in the distal end portion of the pusher catheter 130A, it is possible to prevent the proximal end portion of the stent 70 from being inserted and fitted to the inside of the passage of the hard portion 133, due to an increase of the outer diameter of the hard portion 133 when the proximal d portion of the stent 70 is pushed to the distal end portion of the pusher catheter 130A.

### Third Embodiment

Next, a third embodiment of the present invention will be described with reference to FIG. 14, in which like reference numerals denote like elements, and only differences thereof will be described for the purpose of simplicity.

The delivery system 140 according to the third embodiment illustrated in FIG. 14 has a guide catheter 150 instead of the guide catheter 120 of the delivery system 110 of the second embodiment. The guide catheter 150 has a guide catheter tip portion 61 and a guide catheter body 151. The guide catheter body 151 is formed in a tubular shape similar to the guide catheter body 62 of the first embodiment. Note that, unlike the guide catheter 120 of the second embodiment, the guide catheter body 151 according to the third embodiment does not internally have a reinforcement layer.

The flexural rigidity of the guide catheter 150 can be adjusted appropriately in each part by adjusting shapes such as thicknesses or outer diameters of each part (such as the guide catheter tip portion 61 and the guide catheter body 151) of the guide catheter 150 or by forming the guide catheter 150 by combining different types of materials having different flexural rigidity. For example, if each part of the guide catheter 150 is formed of the same material in an integral manner, the flexural rigidity of the guide catheter 150 can be adjusted by adjusting shapes such as thicknesses or outer diameters of each part. In addition, if the guide catheter 150 is formed by combining different types of materials having different flexural rigidity, for example, the guide catheter tip portion 61 is formed of a polyamide elastomer, and the guide catheter body 151 is formed of a polyamide. In addition, it is possible to obtain the guide catheter 150 having desired flexural rigidity by bonding the guide catheter tip portion 61 and the guide catheter body 151 by heat welding. Further it is possible to adjust the flexural rigidity of the guide catheter 150 by changing an amount of an elastomer added to the polyamide elastomer. In this case, for example, the guide catheter tip portion 61 is formed of a polyamide elastomer containing more of the elastomer than the guide catheter body 151. By bonding the guide catheter tip portion 61 and the guide catheter body 151 as described above, it is possible to obtain the guide catheter 150 having desired flexural rigidity.

Similarly, according to the third embodiment, a stent mounted region Z22 formed by inserting the guide catheter 150 into the passage of the stent 70 and a pusher mounted region Z23 formed by inserting the guide catheter 150 into the passage of the pusher tip 131 of the pusher catheter 130 are set. By adjusting the flexural rigidity in the guide catheter 150, the stent 70, and the pusher catheter 130, the flexural rigidity of the stent 70 becomes equal to or lower than that of the guide catheter body 151, and the flexural rigidity of the stent mounted region Z22 becomes equal to or lower than that of the pusher mounted region Z23.

The operations of the delivery system 140 configured as described above are similar to those of the delivery system 110 of the second embodiment, and thus description thereof will be omitted.

Using the delivery system 140 and the endoscope system according to the third embodiment, similar to the first and second embodiments, it is possible to suppress the aligned portion between the stent 70 and the pusher catheter 130 from being bent with a small radius of curvature.

In the second embodiment, the guide catheter tip portion 61 and the inner layer 122 and the outer layer 123 are formed of the same material as the guide catheter 60 in an integral manner. However, the guide catheter 120 of the second embodiment may be formed of a combination of different materials. That is, for the guide catheter 120 having the inforcement layer 124 interposed between the inner layer 122 and the outer layer 123, it is possible to adjust the flexural rigidity of the guide catheter 120 using a combination of different materials.

While preferred embodiments of the invention have been described and illustrated hereinbefore, it should be understood that they are only for exemplary purposes and are not to be construed as limitations. Any addition, omission, substitution, or modification may be possible without departing from the spirit or scope of the present invention. Further, the elements described in each embodiment may be suitably combined. Accordingly; the invention is not to be considered as being limited by the foregoing description, and is only limited by the scope of the appended claims.

(Supplement 1)

According to the present invention, the guide catheter, the stent, and the pusher catheter preferably have the following relationship in the flexural rigidity. Note that, here, a portion of the guide catheter inserted into the stent is defined as a stent mounted region, and a portion of the guide catheter inserted into the pusher catheter is defined as a pusher mounted region. In the following inequalities, for example, the term “stent” is an abbreviation for “flexural rigidity of stent.”
stent≦guide catheter body
stent mounted region≦pusher mounted region

(Supplement 2)

More preferably the following relationship is established.
guide catheter tip portion<stent

(Supplement 3)

More preferably, the following relationship is established
stent<guide catheter body

(Supplement 4)

More preferably, the following relationship is established.
pusher tip<pusher body

What is claimed is:

1. A stent delivery system comprising:
a guide catheter that can be inserted into a channel of an endoscope;
a stent formed in a tubular shape and provided with a first passage into which the guide catheter can be inserted; and
a pusher catheter formed in a tubular shape, provided with a second passage into which the guide catheter can be inserted, and disposed in a proximal end side relative to the stent,
the guide catheter having, in a state in which the guide catheter is inserted into the stent and the pusher catheter,
a guide catheter tip portion protruding to a distal end side further than the stent,
a guide catheter body disposed inside the stent and the pusher catheter,
wherein a portion of the guide catheter inserted into the stent is defined as a stent mounted region,
a portion of the guide catheter inserted into the pusher catheter is defined as a pusher mounted region,
the stent has flexural rigidity equal to or lower than that of the guide catheter body, and
the stent mounted region has flexural rigidity equal to or lower than that of the pusher mounted region.

2. The stent delivery system according to claim 1, wherein the flexural rigidity of the guide catheter tip portion is lower than that of the stent.

3. The stent delivery system according to claim 2, wherein the flexural rigidity of the stent is lower than that of the guide catheter body.

4. The stent delivery system according to claim 2, wherein the pusher catheter has
a pusher tip and
a pusher body positioned in a proximal end side of the pusher tip, and the flexural rigidity of the pusher tip is to Fe than that of the pusher body.

5. The stent delivery system according to claim 2, wherein the stent has an outer diameter equal to that of a distal end portion of the pusher catheter.

6. The stent delivery system according to claim 4, wherein, in a state the guide catheter is inserted into the stent and the pusher catheter, a distal end side of the guide catheter body protrudes to the distal end side further than the stent.

7. The stent delivery system according to claim 6, wherein the guide catheter tip portion has an outer diameter smaller than that of the guide catheter body.

8. An endoscope system comprising:
an endoscope having
an insertion portion provided with a channel having an opening in a distal end portion, and
a bending section disposed in a proximal end side of the insertion portion relative to the opening and manipulated bendably; and
the stent delivery system according to claim 4, that can be inserted into the channel,
wherein, when at least part of the stent protrudes from the opening of the channel, at least part of the pusher tip is placed inside the bending section in an axial direction of the insertion portion.
